(19) United States
(12) Patent Application Publication
(10) Pub. No.: US 2021/0022722 A1
(43) Pub. Date: Jan. 28, 2021

(54) SURGICAL TOOL

(71) Applicant: Prosys International Limited, London (GB)

(72) Inventor: Gunter Noe, London (GB)

(21) Appl. No.: 16/968,081

(22) PCT Filed: Feb. 6, 2019
(86) PCT No.: PCT/EP2019/052902

§ 371 Date: Aug. 6, 2020

(30) Foreign Application Priority Data
Feb. 6, 2018 (GB) 1801892.9

(51) Int. Cl. A61B 17/02; A61B 17/34; A61B 90/00; A61B 17/00; A61B 90/92

(52) U.S. Cl. CPC A61B 17/0218; A61B 17/3423; A61B 2090/3966; A61B 2017/3425; A61B 2017/00557; A61B 90/92; A61B 2017/0225; A61B 2017/00526

(57) ABSTRACT

A surgical tool (1) for laparoscopic insertion into a subject (13), the tool being expandable from a smaller collapsed configuration to a larger deployed configuration, the tool comprising an extendable barrier (5), and an expandable support (3) that is coupled to the barrier (5); wherein expansion of said support (3) extends said barrier (5).

### FIELD

This invention relates to a surgical tool. In one particularly preferred implementation of the invention the tool is configured for insertion into a subject (for example via a trocar) during laparoscopic surgery (also otherwise known as “minimally invasive” or “keyhole” surgery).

In the following description of aspects of the invention, reference will be made to particular surgical procedures (for example, hysterectomies), but it will be appreciated by persons of ordinary skill in the art that the teachings of the invention are applicable to other surgical procedures, and thus the scope of the present invention should not be limited to the particular surgical procedures described herein.

### BACKGROUND

In laparoscopic surgery it has previously been proposed to create a so-called pneumo-peritoneum in which the abdomen is expanded to thereby facilitate access to internal organs of the subject during the surgical procedure. In one arrangement, it has previously been proposed to pump carbon-dioxide into the abdominal cavity at a controlled pressure, typically at about 12-15 mm mercury.

It has also been proposed, for example for procedures in the lower part of the body, to shift the subject into a so-called “lithotomy” or “Trendelenburg” position in which the body is tilted in a head-down direction by 20 to 45 degrees. For procedures in the upper part of the body (for example, liver, gastric area or gall bladder procedures) it has previously been proposed to tilt the subject into a feet-down position known as a reverse Trendelenburg position.

These subject positions are adopted to facilitate the surgeon's access to the intended area of interest. In the context of pelvic surgery, the bowel (which moves continuously) is in danger of being injured by needles, scalpels, scissors or hot instruments during a given procedure. By adopting a Trendelenburg position for the subject, gravity assists the surgeon by tending to draw the bowel and other organs towards the head of the subject and thus out of the surgeon's way.

Obesity can reduce the effectiveness of the Trendelenburg position, as the nutritional area of the bowel tends to have more fatty tissue that hinders displacement of the bowel and other organs. It is also the case that the pressure of a higher fat load on the internal abdominal wall of obese subjects further hinders the effectiveness of the Trendelenburg position. The foregoing issues can complicate pelvic surgical procedures, and in some instances make such procedures entirely unfeasible.

To address such issues, it has previously been proposed to adopt an extreme Trendelenburg position (i.e. to increase the degree to which the subject is tilted), but such a position greatly increases the pressure in the head of the subject with potentially severe consequences. In addition, such a position can endanger subjects with vascular risks, as well as those with multiple co-morbidities (for example, blood pressure problems, reduced kidney function etc.).

If the Trendelenburg position is not sufficient to keep the bowel and other organs out of the surgeon's way or an extreme Trendelenburg position is not feasible due to the morbidity of the patient, the bowel has to be retracted by a grasper or retractor tools. Since the latter are only available as 10 mm instruments, larger trocars must necessarily be inserted into the subject. Graspers also increase the risk of inadvertently harming the bowel during the procedure as the tool will typically be out of sight.

As mentioned briefly above, extreme Trendelenburg positions can lead to many side effects. Redistribution of pooled venous blood from the lower limbs tends to increase venous return to the heart and thus increases cardiac output. This increased cardiac output can partially offset the cardiovascular depressant effects of many anaesthetic techniques. It is also the case that complications due to airway obstruction and decreased tidal volumes can occur.

For obese or pregnant subjects, severe hypotension may occur as a result of compression of the inferior vena cava against the vertebral bodies. In such circumstances, central redistribution of blood may lead to volume overload in the failing heart.

It is also the case that in supine patients, there is an increased risk of regurgitation of gastric contents, and diaphragmatic movement can be limited severely by the weight of the abdominal viscera which further reduces FRC (functional respiration capacity) and increases atelectasis. Ventilation-perfusion mismatch, raised intracranial pressure, raised intraocular pressure and passive regurgitation are all potential complications; the severity and likelihood of which increase with the amount of tilt.

As obesity is increasing in society, problems of the type aforementioned have become more commonplace. Aspects of the present invention have been devised with such problems in mind.

### SUMMARY

In accordance with a presently preferred embodiment of the present invention, there is provided a surgical tool for laparoscopic insertion into a subject, the tool being expandable from a smaller collapsed configuration to a larger deployed configuration, the tool comprising an extendable barrier, and an expandable support that is coupled to the barrier; wherein expansion of said support extends said barrier.

Preferably the support extends around at least part of the periphery of said barrier. The support preferably extends around the entire periphery of the barrier. The support preferably defines an aperture, the barrier being located in said aperture. The support is preferably annular, and the tool is generally circular or oval in said expanded configuration. Preferably the tool is suitable for insertion into a subject via a trocar when in said collapsed configuration. The support and barrier may be folded, furled or rolled together in said collapsed configuration. The support may includes an internal void, and the tool may comprise a lumen having a distal end and a proximal end, said distal end being coupled to said support and opening to said void.

The support may be expanded by injecting fluid into said void via said lumen. The support may be inflatable via said lumen. The proximal end of said lumen remote from said support may include a coupling for connecting said lumen to a source of fluid for injection into said lumen. The coupling may include a valve.

The barrier may comprise a single layer of flexible material, or two adjacent layers of flexible material.

The tool is preferably comprised of a biocompatible material. At least said barrier may be comprised of a material that is cut resistant and/or heat resistant.

Another aspect of the teachings of the invention comprises a surgical kit comprising: a surgical tool as described herein, and a trocar by means of which the tool can be laparoscopically inserted into a subject whilst the tool is in said collapsed configuration. The kit may include a syringe that is connectable to said support and is operable to expand said support. The kit may include an applicator for inserting the tool into a subject.

Another aspect of the teachings of the invention relates to a method of surgery comprising the steps of: making an incision in a subject to provide access to the subject's body cavity; inserting a tool as described herein into said body cavity, and expanding said tool from said collapsed configuration into said larger deployed configuration.

A yet further aspect of the teachings of the invention provides a method of manufacturing a tool of the type described herein, comprising the steps of: forming a first component comprising a first barrier and a first half support extending around the first barrier; forming a second component comprising a second barrier and a second half support extending around the second barrier; and coupling the first and second components together so that the first and second barriers are adjacent and the first and second half supports form an expandable tubular body.

In another aspect, there is provided a method of manufacturing a tool of the type described herein, comprising the steps of: forming a first component comprising a barrier and a first half support extending around the barrier; forming a second component comprising an annular second half support; and coupling the first and second components together so that the first and second half supports form an expandable tubular body surrounding said barrier.

A further aspect provides a method of manufacturing a tool of the type described herein, comprising the steps of: forming a barrier, forming an annular tubular support, and coupling the barrier to the support so that the support extends around the periphery of the barrier.

In one implementation, there is provided a surgical tool for laparoscopic insertion into a subject (optionally) via a trocar, the tool being expandable from a smaller collapsed configuration to a larger expanded (deployed) configuration, the tool comprising an extendable barrier, and an expandable support that is coupled to the barrier; wherein expansion of said support to extend and thereby deploy the barrier causes the tool to move from the collapsed configuration towards the expanded (deployed) configuration.

By adopting the teachings of the present invention, the tool can be used to keep the bowel and other organs out of the operative area and thereby facilitate access for the surgeon. The surgeon can work more effectively (saving time), and the risk of injury to the bowel can be reduced. The teachings of the invention also provide a tool that enables the surgeon to reduce the Trendelenburg position, thereby lowering general risks for subjects, and making surgery more available to high risk patients for whom such surgery was not previously feasible.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is a schematic representation of a surgical tool in a collapsed configuration;

FIG. 2 is a schematic representation of the tool depicted in FIG. 1 in a deployed configuration;

FIG. 3 is a diagrammatic sectional view of a supine subject with the tool of FIG. 2 inserted;

FIG. 4 is a partly cut-away diagrammatic plan view of the subject depicted diagrammatically in FIG. 3; and

FIGS. 5 to 10 are schematic plan views of further tools that embody the teachings of the invention.

### DETAILED DESCRIPTION

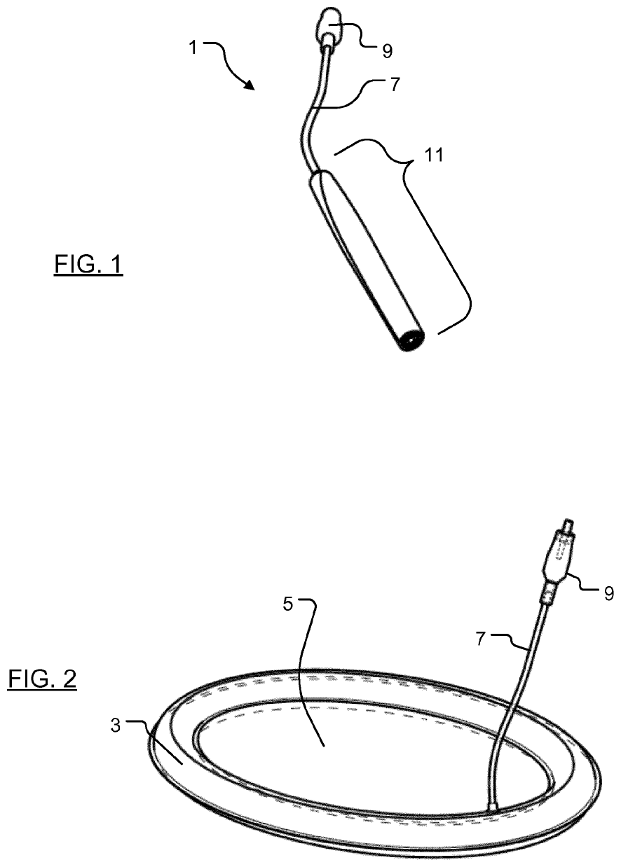

FIGS. 1 and 2 of the accompanying drawings are diagrammatic representations of a surgical tool 1 that embodies the teachings of the invention in a collapsed and an expanded configuration, respectively.

Referring now to FIGS. 1 and 2, the tool 1 comprises an expandable support 3 that is coupled to, and in this implementation, surrounds an extendable barrier 5. The expandable support comprises a tubular body that is in fluid communication with a lumen 7 which terminates at a fluid coupling 9. The fluid coupling 9 allows the lumen 7 (and hence the support 3) to be connected to a fluid pump (not shown) so that fluid can be pumped into the lumen to expand the support 3, and extend—and optionally tension—the barrier 5. The tool 1 is preferably circular when the support has been expanded and the barrier extended, but may conceivably be oval.

In one envisaged arrangement, the tool may be configured so that it can be unfurled by the surgeon whilst located inside the patient, and to this end may include one or more tabs (not shown) (for example coloured tabs that can more easily be located by the surgeon) that facilitate grasping of the tool by the surgeon using conventional laparoscopic tools. In another envisaged arrangement, the tool may be configured to automatically unfurl to extend the barrier as fluid is pumped into the extendable barrier. In yet another arrangement, the surgeon may partly unfurl the tool before expanding the support to extend the barrier. The tool may include one or more radio-opaque markers (not shown) to facilitate imaging of the tool within the subject.

In one envisaged implementation, the coupling may include a valve so that the fluid pump can be decoupled from the lumen once the support has been expanded. In another envisaged implementation, the fluid coupling may be without a valve and simply comprise a means for coupling the fluid pump to the support so that fluid may be pumped into and out of the support.

The support 3, barrier 5 and lumen are of a resilient, flexible biocompatible material, for example of silicone. At least the barrier 5 may be of a biocompatible material that is cut resistant and/or heat resistant. As shown in FIG. 1 the support 3 and barrier 5 can be rolled up into a cylindrical body 11 that is sized so that it can be inserted into the peritoneum of a subject, for example via a trocar. Alternatively, the surgeon may opt to manually poke the rolled up tool into the subject through an existing or new incision, for example an incision in the umbilicus. In one envisaged mode of operation, once the tool has been inserted and the fluid coupling 9 has been connected to a fluid pump, the pump can operated to pump fluid through the coupling 9 and into the lumen 7, whereupon the support 3 will start to expand and the cylindrical body will start to unfurl (and thereby starts to extend and deploy the barrier 5). Operation of the pump is continued until the tool 1 assumes the shape depicted in FIG. 2. In another envisaged mode of operation the tool may be partly or fully unfurled by the surgeon (using conventional laparoscopic tools) before the pump is operated to expand the support and extend the barrier.

In the preferred implementation, the support 3 is inflated by pumping a gas, for example Carbon Dioxide or Nitrogen, into the lumen 7, but it will be appreciated that other gases or fluids could instead be pumped into the support to extend and deploy the barrier 5.

In one envisaged mode of operation, at the start of a surgical procedure in which the tool 1 is to be used, the surgeon performing the procedure inserts a tool as depicted in FIG. 1 into the subject's inflated abdomen (for example via a trocar) so that the fluid coupling (and optionally some of the lumen) lies outside of the subject's body. The surgeon may then use conventional laparoscopic techniques to position the tool, and once properly positioned the surgeon can operate a fluid pump coupled to the fluid coupling to expand the support and thereby extend the barrier within the subject's peritoneum. The surgeon can then press the subject's internal organs aside (for example, by using a flat spatula), before placing the tool against the subject's organs so that those organs are kept clear from the area of the subject that the surgeon is going to operate on. To facilitate this part of the procedure, the surgeon may place the patient in the aforementioned Trendelenburg position, but in a departure from previous proposed techniques, once the surgical tool has been expanded and properly positioned within the subject, the extent to which the subject is inclined in the Trendelenburg position can be reduced and in some instances the subject can be returned to a conventional supine position.

In another mode of application, the surgeon may use conventional laparoscopic techniques to unfurl the tool within the patient, whereupon the support can then be expanded by means of the lumen that is at least partly outside of the subject's body. In yet another mode of application, the surgeon may insert the tool and then use conventional laparoscopic techniques to partly unfurl the tool, before expanding the support to extend the barrier.

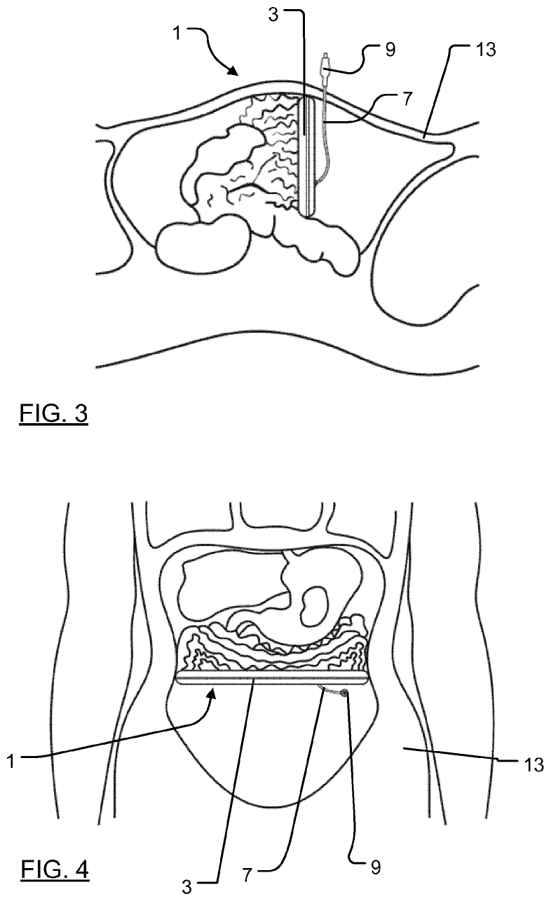

When positioned within the subject's body and in the expanded configuration, the internal pressure of the pneumo-peritoneum bears against the tool and helps keep the tool in a position where the subject's internal organs are displaced from the region in which the surgeon is to conduct the procedure. FIGS. 3 and 4 are schematic cross-sectional side and part cut-away plan representations, respectively, of a subject 13 in whom a tool 1 has been inserted, unfurled, positioned and expanded/extended. When so arranged, the aforementioned Trendelenburg position can at least be reduced or in some instances altogether avoided.

More generally, it is the case that obstacles such as a moving bowel tend to be problematic during surgery. This is especially the case in laparoscopic surgery, in many different fields. Since many gynaecological procedures are performed by laparoscopy it is anticipated that the teachings of the present invention will have particular application in this field as such procedures previously tended to require that the patient is in a head down position in order to provide access to the region and the organs.

Illustrative procedures in which the tool may be employed are: Adnex removal, Tubal reconstruction, Adnex-cyst removal, ovary reconstruction, Hysterectomy (sub-total; total; and radical), Pelvic Lymphadenectomy, Fibroid resection, Uterus reconstruction, Colposuspension; Sacropexy; Pectopexy; laparoscopic lateral repair; laparoscopic anterior facia repair; laparoscopic posterior fascia repair; Laparoscopic recto-pexy; laparoscopic CESA/VASA; Peritoneal resection in the pelvis; Endometrioma removal; Bowel resection; ureter resection; de-bulking; adhesilyses; ureterolysis; bladder resection; ureterolysis; ureter implantation; Psoas-Hitch procedure; prostatectomy, urethra surgery; pelvic lymphnodes, Radical Hysterectomy; pelvic and para-aortal lymphadenectomy; radical resection of ovarian cancer; rectum cancer resection; sigmoid-resection, Rectum resection; and sigmoid resection.

In laparoscopic procedures, it is usually the case that the umbilicus is used to provide access for a camera as it is a natural scar and the smallest point of the abdominal wall. According to the preference of the surgeon 0-45 degree scopes with a camera are used to monitor the surgery via a display. Access ports of different diameter (5-10 mm) are then placed by the surgeon in the lower abdomen. The placement of these ports depends on the predilections of the surgeon or special requirements of the procedure being undertaken.

One illustrative gynaecological procedure is the hysterectomy. This procedure starts with an incision in the umbilicus and dilatation of the fascia to provide a blind opening of the peritoneum. Next, a 10 mm trocar is placed in the incision, and an insufflation tube (for example, CO₂) is connected. The peritoneum is then inflated under pressure control (12-15 mmHg) (approximately 1.8-3 litre CO₂), and a camera is introduced into the 10 mm trocar so that the surgeon can visually inspect the complete abdominal area. The patient is then put into the Trendelenburg position (15-45 degrees) and access-ports are introduced in the lower abdomen. Usually two or three 5 mm ports are inserted. The leading surgeon for example may then take a blunt grasper in his left hand and a coagulation grasper in his right hand. The assistant may have an additional grasper in his right hand and may control the camera with his left.

The procedure proper starts by heating the lateral upper ligaments (round ligament), after which the surgeon can cut the tissue with a scissor without bleeding. The peritoneum is then opened anteriorly in the direction of the bladder, and the connective tissue between the ovary and the uterus (ligament) is heated before being cut so that the ovary can be pushed away. The posterior peritoneum is then cut and opened completely, and the aforementioned steps are repeated for the other ovary.

The surgeon then locates the arteries that supply the region with blood and surrounding tissue is removed. The arteries are then heated and subsequently cut, before lower tissue is heated and dissected. The cup surrounding the cervix in the vagina is then pushed towards the surgeon so that the surgeon can identify the cup by touch.

The uterus is then pealed out of the lower connective tissue with a small hook. Monopolar energy heats the hook so that the surgeon can cut the tissue without causing bleeding. The dissection is performed straight to the rim of the cup to enable a close resection of the uterus without losing too much vaginal or connective tissue. The uterus is then removed vaginally

The vagina is then filled with a sponge or a small towel to avoid leakage of gas from the pneumo-peritoneum, and the vaginal cuff is closed by a suture (by single knots or a running suture). The area is then cleaned by irrigation and suction, following which the surgical field, bowel and both sides of the ureter are inspected.

Next the surgeon removes the access trocars, deflates the pneumo-peritoneum, and removes the camera port. The surgeon then sutures the fascia in the umbilicus, and the lower and umbilical incisions. Lastly, the surgeon disinfects the area (including the vagina) before applying sterile bandages.

In a typical hysterectomy procedure, the subject is in the Trendelenburg position for much of the procedure. By utilising a tool according to the teachings of the present invention, once the tool has been inserted and positioned the Trendelenburg position can be reduced, in some instances to zero.

Surgery in the upper part of the abdomen requires a reverse Trendelenburg position of the patient. Due to the presence of heavy fatty tissue obese subjects can be particularly difficult to operate on. A tool of the type described herein can improve access to the organs in this area. Endometriosis and some oncological procedures in gynaecology can also require surgery in the upper area. The tool described herein may also be useful in visceral surgery, for example in cholecystectomy; liver resection; gastric resection, diaphragm hernia, Omental resection and Spleen removal.

The tool described herein may be manufactured in a variety of different ways. In one envisaged arrangement, the support and barrier may be separately formed (for example by moulding), and then joined to one another (for example by welding). However, since separately moulding the two components may prove problematic, it is also envisaged to form the barrier and half of the support as a first component and the remaining half of the support as a second component that is then joined to the half support of the first component. In yet another arrangement it is proposed to form the tool as two components that each comprise a barrier arranged between two half supports, before subsequently joining the half support of one component to the half support of the other. An advantage of this last arrangement is that the tool would then comprise two adjacent barriers, and thus would be more resistant to inadvertent puncture by the surgeon than a tool with a single barrier.

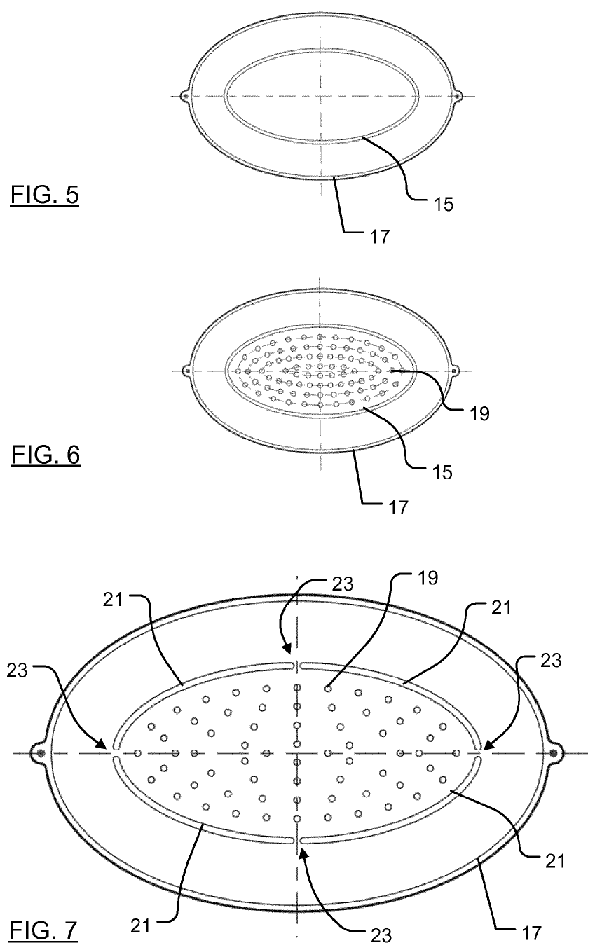

In the context of this last arrangement, a further advantage of having two components is that the components may be coupled together in a variety of different ways. For example, as depicted in FIG. 5, the two components could simply be coupled together by means of an inner weld line 15 and an outer weld line 17 to create a tubular body extending therebetween (which body can be inflated as previously described). In another envisaged arrangement depicted schematically in FIG. 6, the aforementioned inner and outer weld lines may be supplemented by a plurality of spot welds 19 which couple the two parts of the barrier together and help reduce the likelihood of the tool twisting as the support is inflated.

In a further arrangement, shown schematically in FIG. 7, four inner weld lines 21 are provided, with each said weld line being separated from adjacent weld lines so as to provide a channel 23 therebetween. As will be appreciated by persons skilled in the art, when the tool is inflated, fluid can pass through the aforementioned channels and into the space between the two parts of the barrier that are not coupled together by spot welds 19 to inflate the barrier and thereby reduce the likelihood of the tool twisting as it is inflated.

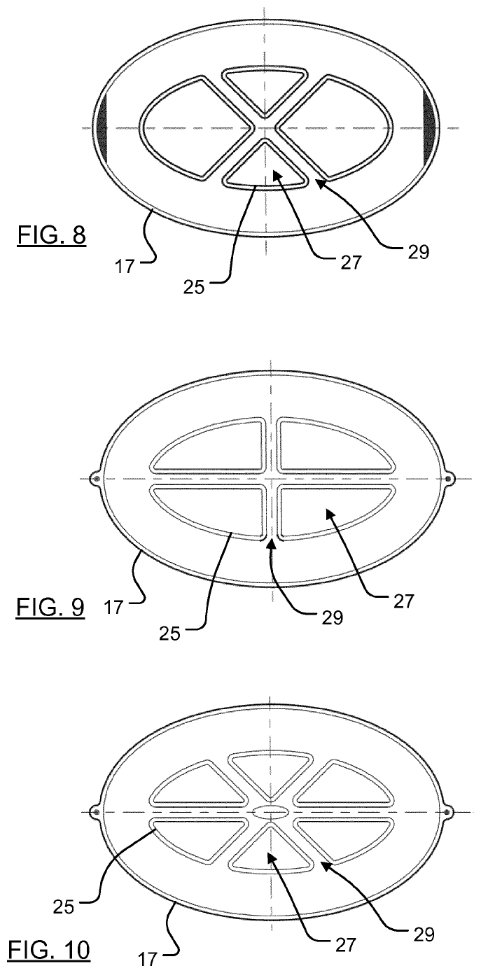

In other arrangements depicted schematically in FIGS. 8 to 10, a plurality of inner weld lines 25 may be provided, which weld lines divide the barrier into a plurality of regions 27 that are separated from one another by channels 29 which criss-cross through the barrier from one part of the support to a diametrically opposite part of the support, in a manner akin to the spokes of a bicycle wheel. In a similar manner to the arrangement shown in FIG. 7, when the tool is inflated, the channels 29 also inflate and thereby increase the tool's torsional rigidity to reduce the likelihood of the tool twisting as it is inflated. FIGS. 8, 9 and 10 show, respectively, tools with two, two and three channels, although it will be appreciated that the number of channels may be varied as desired. In addition, it is also conceivable for the aforementioned regions 27 to include a plurality of spot welds, for example as shown in FIGS. 6 and 7.

The tool may be packaged in its rolled up form (i.e. as shown in FIG. 1), or in another envisaged arrangement the tool may be configured for use with an insertion device, for example a device akin to a conventional tampon applicator, and may be provided packaged with such a device ready for insertion into a subject.

It will be appreciated that whilst various aspects and embodiments of the present invention have heretofore been described, the scope of the present invention is not limited to the particular arrangements set out herein and instead extends to encompass all equivalents, arrangements, and modifications and alterations thereto, which fall within the scope of the appended claims.

It should also be noted that whilst the accompanying claims set out particular combinations of features described herein, the scope of the present invention is not limited to the particular combinations hereafter claimed, but instead extends to encompass any combination of features herein disclosed.

Finally, it should be noted that any element in a claim that does not explicitly state “means for” performing a specified function, or “steps for” performing a specific function, is not to be interpreted as a “means” or “step” clause as specified in 35 U.S.C. Sec. 112, par. 6. In particular, the use of “steps of” in the claims appended hereto is not intended to invoke the provisions of 35 U.S.C. Sec. 112, par. 6.

1-23. (canceled)

24. A surgical tool for laparoscopic insertion into a subject, the tool comprising:
an extendable barrier; and
an expandable support coupled to the extendable barrier; wherein expansion of the support extends the barrier, thereby expanding the tool from a smaller collapsed configuration to a larger deployed configuration.

25. The surgical tool according to claim 24, wherein the support extends around at least part of a periphery of the barrier.

26. The surgical tool according to claim 25, wherein the support extends entirely around the periphery of the barrier.

27. The surgical tool according to claim 24, wherein the support defines an aperture, the barrier being located in the aperture.

28. The surgical tool according to claim 27, wherein the support is annular and the tool is generally circular or oval in the deployed configuration.

29. The surgical tool according to claim 24, wherein the tool is suitable for insertion into the subject via a trocar when in the collapsed configuration.

30. The surgical tool according to claim 29, wherein the support and barrier are folded, furled, or rolled together in the collapsed configuration.

31. The surgical tool according to claim 24, wherein the support includes an internal void and the tool comprises a lumen having a distal end and a proximal end, the distal end being coupled to the support and opening to the void.

32. The surgical tool according to claim 31, wherein the support can be expanded by injecting fluid into the void via the lumen, and/or the support is inflatable via the lumen.

33. The surgical tool according to claim 31, wherein the proximal end of the lumen remote from the support includes a coupling for connecting the lumen to a source of fluid for injection into the lumen.

34. The surgical tool according to claim 24, wherein the barrier comprises a single layer of flexible material or two adjacent layers of flexible material.

35. The surgical tool according to claim 24, wherein at least the barrier is comprised of a material that is cut resistant and/or heat resistant.

36. The surgical tool according to claim 24, further comprising a trocar via which the tool can be laparoscopically inserted into the subject whilst the tool is in the collapsed configuration.

37. The surgical tool according to claim 36, further comprising a syringe that is connectable to the support and is operable to expand the support.

38. The surgical tool according to claim 36, further comprising an applicator for inserting the tool into the subject.

39. A method of surgery, the method comprising:
making an incision in a subject to provide access to a body cavity of the subject;
inserting a tool into the body cavity, the tool comprising
an extendable barrier; and
an expandable support coupled to the extendable barrier; wherein expansion of the support extends the barrier, thereby expanding the tool from a smaller collapsed configuration to a larger deployed configuration; and
expanding the tool from the collapsed configuration into the larger deployed configuration.

40. A method of manufacturing a surgical tool, the method comprising:
forming a first component comprising a first barrier and a first half support extending around the first barrier;
forming a second component comprising a second barrier and a second half support extending around the second barrier; and
coupling the first and second components together so that the first and second barriers are adjacent and the first and second half supports form an expandable tubular body.

41. A surgical tool for laparoscopic insertion into a subject's abdomen, the tool comprising:
an extendable barrier comprising two adjacent layers of flexible material that are coupled to one another to form one or more channels between the layers of flexible material; and
an expandable support in fluid communication with the channels and coupled to the extendable barrier, wherein expansion of the support extends the barrier and expands the channels to enhance torsional rigidity of the tool and thereby expanding the tool from a smaller collapsed configuration to a larger deployed configuration.
